(19) United States
(12) Patent Application Publication
(10) Pub. No.: US 2007/0142367 A1
(43) Pub. Date: Jun. 21, 2007

(54) METHOD AND MEDICINE FOR TREATING GASTROINTESTINAL DISORDER INCLUDING FECAL INCONTINENCE

(76) Inventor: James E. Lundeen, Cleveland Heights, OH (US)

Correspondence Address: Thomas W. Adams;RENNER, OTTO, BOISSELLE & SKLAR, LLP, 19th Floor, 1621 Euclid Avenue, Cleveland, OH, 44115, US

(21) Appl. No.: 11/671,298

(22) Filed: Feb. 5, 2007

Related U.S. Application Data
(63) Continuation-in-part of application No. 10/970,501, filed on Oct. 21, 2004, now abandoned.
(63) Continuation-in-part of application No. 10/970,149, filed on Oct. 21, 2004, now abandoned.
(60) Provisional application No. 60/518,715, filed on Nov. 10, 2003.
(60) Provisional application No. 60/518,718, filed on Nov. 10, 2003.
(60) Provisional application No. 60/518,719, filed on Nov. 10, 2003.
(60) Provisional application No. 60/518,715, filed on Nov. 10, 2003.
(60) Provisional application No. 60/518,718, filed on Nov. 10, 2003.
(60) Provisional application No. 60/518,719, filed on Nov. 10, 2003.

(51) Int. Cl. A61K 31/5415; A61K 31/185

(52) U.S. Cl. 514/225.2; 514/553

(57) ABSTRACT

A method and a medicine for treating an ambulatory patient having a gastrointestinal disorder that includes fecal incontinence are provided. The method includes administering a dose of the medicine to the patient. The medicine includes a tricyclic antidepressant and a stool softener.

### CROSS-REFERENCE TO RELATED APPLICATIONS

This application is a continuation in part of and claims priority under 35 U.S.C. 120 to copending U.S. application Ser. No. 10/970,501, filed 21 Oct. 2004 and to copending U.S. application Ser. No. 10/970,149, filed 21 Oct. 2004; both of which in turn claim priority under 35 U.S.C. 119(e) to U.S. Provisional Patent Applications Ser. Nos. 60/518,715 filed on Nov. 10, 2003; 60/518,718 filed on Nov. 10, 2003; and 60/518,719 filed on Nov. 10, 2003, the disclosures of all of which are hereby incorporated by reference in their entirety.

### BACKGROUND

1. Field of Invention

The present invention generally relates to a method and a medicine for reducing or eliminating the undesirable affects of a gastrointestinal disorder. The present invention relates to a method for reducing or eliminating fecal incontinence associated with the gastrointestinal disorder. The present invention relates to a medicine for reducing or eliminating fecal incontinence associated with the gastrointestinal disorder.

2. Discussion of Related Art

Gastrointestinal disorders may manifest with one or a plurality of symptoms, including fecal incontinence. Fecal incontinence may be either an acute functional disorder or a chronic functional disorder of the lower bowel. An acute attack may last only a few days or weeks, and is not recurring over a long period of time. In contrast, a chronic affliction may last or persist for a long time, or may reoccur regularly or irregularly over a long time.

The walls of the gastrointestinal (GI) tract have four layers: the mucosa, submucosa, muscluaris externa, and serosa. The mucosa consists of an epithelium with basement membrane (called the lamina propria), loose connective tissue, blood vessels, and lymph tissues. The submucosa contains loose connective tissue, glands, nerves, and blood vessels. The nerve fibers of the submucosa form a network or plexus called the plexus of Meissner. The muscularis externa may include two bands of smooth muscle cells, the internal layer is composed of circular smooth muscle and the external layer is composed of longitudinal fibers. Interspersed between the muscle fibers is a nerve plexus called the plexus of Auerbach. The outermost layer of the digestive tube, the serosa, is composed of a membrane of squamous epithelium.

The gastrointestinal tract has a simplified “brain”, or nerve network, in the myenteric and submucosal plexuses, which has about 100 million neurons (the enteric nervous system). Efferents in the submucosal Meissner plexus regulate secretion by intestinal glands. The efferents in the myenteric Auerbach plexus control peristalsis, which is the rhythmic contraction of circular and longitudinal muscles. Visceral sensory afferent nerve endings are located throughout the submucosa and the Meissner plexus. Cranial nerves of the parasympathetic nervous system (e.g., the vagus) convey much of the sensory information from the gastrointestinal tract. However, sympathetic afferent nerves may transmit visceral sensations of pain to the spinal cord. Sensations, motility, digestion and secretion may be controlled by nerve elements of the gastrointestinal tract. The gastrointestinal tract submucosa contains sensory afferent nerve fibers that code for pressure, temperature and pain signals. Injury to the gastrointestinal tract may contribute to gastrointestinal disorders, such as fecal incontinence.

Treatment options for fecal incontinence may range from dietary modification and drug therapy to surgery. With respect to diet, the patient may avoid foods to which they possess a known sensitivity with respect to exacerbating the problem.

With reference to drugs, medicines and other treatments for fecal incontinence, few or none have demonstrated sufficient efficacy or sustainability to be of practical benefit to a majority of patients. Drugs and medicines directed to the gastrointestinal tract may include antacids, anti-spasmodic agents, anti-diarrheal drugs, anti-inflammatory drugs such as glucocorticosteroids and NSAIDS, histamine-R2-blocking agents, and antibiotics. Treatment may include surgery of the affected tissues, which may be an undesirable option.

Anti-spasmodic (anti-cholinergic) medication may be prescribed to decrease intestinal motility. U.S. Pat. Nos. 4,611,011, 4,701,457, and 4,745,131 disclose a series of amidinoureas which reduce intestinal motility. 1-Azabicyclo [2-2-2] octan-3-yl-2-aryl-3-azacyclo-2-hydroxy propionates and their quaternary salts, which possess antispasmodic activity, are disclosed in U.S. Pat. No. 4,843,074. Calcium channel antagonists exhibit muscle relaxing and antispasmodic activities. A series of substituted imidazolyl-alkyl-piperazine and diazepine derivatives, disclosed in U.S. Pat. No. 5,043,447, are calcium channel antagonists and may be useful as antispasmodics. U.S. Pat. No. 4,877,779 discloses 2-Aminomethylalkynylalkyl-1,3-dithiane derivatives with calcium-channel blocking activity and potentially similar uses. Some triazinone derivatives having spasmolytic activity are disclosed in U.S. Pat. No. 4,562,188.

In addition to antispasmodic agents, compounds with other activities have been disclosed which may relieve symptoms. Anti-diarrheal agents, such as loperamide, diphenoxylate, and codeine phosphate, have been used. Unfortunately, such agents are of little practical long-term benefit. Other anti-diarrheals include anti-cholinergics and smooth muscle relaxants, such as cimetropium bromide, pinaverium bromide, octilium bromide, trimebutine, and mebeverine.

Anti-spasmodics and anti-diarrheal preparations may be used to treat fecal incontinence. Even if effective, use of these preparations for long-term treatment may be precluded by problems such as development of tolerance, toxicity, or abuse potential.

Non-selective excitatory opioid receptor antagonists have been identified as central nervous system treatments that affect various gastrointestinal functions, and have been used to treat gastrointestinal disorders. Non-selective excitatory opioid receptor antagonists include, for example, tricyclic antidepressants, such as amitriptyline, imipramine, and doxepin. These opioid receptor antagonists may be effective for some gastrointestinal disorder symptoms due to the neuromodulatory and analgesic properties of these compounds. However, the administration of the receptor antagonists has been precluded for long-term care for chronic conditions.

While the manufacturer's recommended dosages of imipramine pamoate may be modified as necessary, the manufacturer's recommended dosages include: initial adult dosage for outpatients starting at 75 mg/day, which may be increased to 150 mg/day—the level at which the optimum response is usually obtained for anti-depression treatment. For anti-depression treatment, the manufacturer's recommended dosages for hospitalized patients start at an even higher dose of 100-150 mg/day—and the dosage can be raised as high as 300 mg/day. Elderly patients and children are stated as likely to respond to a dosage of about 25-50 mg/day per manufacturer's recommendations.

In contrast to the non-selective antagonists above, selective excitatory opioid receptor antagonists have been studied as treatments for gastrointestinal disorders. For example, U.S. Pat. No. 5,512,578 discloses co-administration of a selective excitatory opioid receptor antagonist with bimodally-acting opioid agonist, which may enhance potency of an analgesic, and may reduce tolerance and dependence liability. Such selective antagonists include, when administered at appropriately low doses, naloxone, naltrexone, etorphine, and dihydroetorphine. The selective excitatory opioid receptor antagonists attenuate excitatory, but not inhibitory, opioid receptor functions in nociceptive (pain) pathways of the peripheral and central nervous systems. As a result, symptoms associated with activation of excitatory opioid receptors, such as anti-analgesia, hyperalgesia, hyperexcitability, physical dependence and/or tolerance effects may be increased.

In spite of the many treatments, compositions and methods used to reduce or eliminate fecal incontinence, a suitable long-term efficacious treatment or preventative has not been identified. It may be desirable to have a medicinal composition or medicine having improved properties for the treatment of fecal incontinence and/or fecal urgency. It may be desirable to have improved or alternative methods of treatment for the treatment of fecal incontinence and/or fecal urgency.

### SUMMARY

The present invention relates to a method for treating a human having a gastrointestinal disorder. The disorder may include fecal incontinence and/or fecal urgency. The method includes administering a dose of the medicine to the human. The medicine includes a tricyclic antidepressant and a stool softener.

An embodiment of the present invention relates to the medicine for treating a human having a gastrointestinal disorder that includes fecal incontinence. The medicine includes a tricyclic antidepressant and a stool softener.

In one embodiment, the invention relates to a process that includes interacting with muscarinic receptors in the human to reduce or eliminate fecal incontinence and/or fecal urgency. In one embodiment, the process further includes emulsifying oil and water into fecal matter using the surfactant to soften the stool of the human, lubricating the fecal matter to facilitate passage of the stool, or both emulsifying and lubricating the fecal matter to both soften the stool and facilitate passage of the stool.

### BRIEF DESCRIPTION OF THE DRAWINGS

FIG. 1 is a schematic diagram showing a packaging configuration of a medicine comprising an embodiment in accordance with the invention; and

FIG. 2 is a schematic block diagram showing a method in accordance with the invention.

### DESCRIPTION OF PREFERRED EMBODIMENTS

The present invention relates to a method of treating a chronic disorder of the gastrointestinal (GI) tract with a medicinal composition. In one embodiment, the present invention relates to a method of treatment. In another embodiment, the present invention relates to a medicinal composition.

As used herein, a chronic condition refers to a condition that lasts for a substantial period or long time, and in some instances a chronic condition may not have an endpoint. Furthermore, chronic conditions may be continuous or recurring, and may reoccur regularly or irregularly. Gastrointestinal tract disorders include encopresis (fecal incontinence) and/or diarrhea. Diarrhea is intended to broadly include both a medical practitioner's definition—an increased frequency of bowel movements, and a lay person's definition—liquid or fluid stool that causes difficulty of continence. A medicinal composition (“medicine”) is a substance administered in the treatment of a disorder; a remedial agent or a remedy; and a preventative. An efficacious amount is an amount greater than zero that has a desired or desirable effect.

Methods according to embodiments of the invention include the administration of a dose or a series of doses of the medicine to an ambulatory patient suffering from or presenting symptoms associated with a gastrointestinal (GI) disorder. Dosage information is described below. The gastrointestinal disorder may include fecal incontinence and/or fecal urgency, and may be a result of, for example, one or more of nerve injury, a course of radiation treatments, a hemorrhoid surgery, a chemotherapy treatment, a compromised vascular supply to the bowel, malnutrition, diabetes, cancer, or other, possibly unknown, sources. The nerve injury may be, for example, a spinal nerve injury, a spinal cord injury, or a pelvic nerve injury. The compromised vascular supply to the bowel may be a result of, for example, cigarette smoking, a high cholesterol condition, collagen vascular disease, or a stroke of the bowel mesenteric artery.

The present invention is applicable to a wide variety of patients. In one embodiment, the patient treated in accordance with the invention is ambulatory, that is, the patient is able to walk about, and is not confined to bed or hospital, e.g., as a result of injury, disease or surgery. In one embodiment, the patient treated in accordance with the invention is a person not confined to a nursing home. In one embodiment, the patient treated in accordance with the invention has suffered a spinal injury. In one embodiment, the patient treated in accordance with the invention has had spinal surgery. Many patients with spinal injuries have bladder urgency, frequency, stress incontinence, post-void incontinence and/or continuous incontinence. Many patients with spinal injuries have fecal urgency, fecal incontinence, fecal stress incontinence and/or incontinence of flatus, whether or not the stool is liquid, semi-solid or formed. Such patients generally do not suffer from fecal impaction. In one embodiment, the patient treated in accordance with the invention does not suffer fecal impaction. In one embodiment, the fecal incontinence treated by the method of the present invention does not arise as a result of fecal impaction. In one embodiment, the patient treated in accordance with the invention is not being treated with a pharmaceutical containing a narcotic agent. In other embodiments, the patient treated in accordance with the invention also is under treatment with a pharmaceutical containing a narcotic agent.

In one embodiment, the medicine includes a tricyclic antidepressant and one or both of a stool softener. Tricyclic antidepressants may be used alone or in combination and may include amitriptyline, clomipramine, desipramine, imipramine, doxepin, and nortriptyline, and derivatives and pharmaceutically acceptable salts thereof. Unless otherwise specified or indicated by context, “stool softener” will herein collectively include both stool softener and fecal lubricant for ease of referral. The medicine according to embodiments of the present invention may be used to treat fecal incontinence. The stool softener (that may increase stool frequency) is present regardless of frequent stools.

In one embodiment, the tricyclic antidepressant includes imipramine (5-[3-(dimethylamino)propyl]-10,11-dihydro-5H-dibenzazepine), which is shown structurally below, or an active metabolite thereof—such as desmethylimipramine.

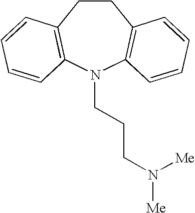

In another embodiment, the tricyclic antidepressant includes imipramine HCI. Imipramine hydrochloride is available for commercial sale as TOFRANIL from Mallinckrodt Inc. (St. Louis, Mo.). As used throughout, reference to dosage of imipramine generally will be to an equivalent amount of imipramine HCI. Also, unless specified, dosage values are in units of milligrams. For anti-depression treatment, manufacturer's supply TOFRANIL in 10, 25, 50 and 75 mg tablets or capsules.

In one embodiment, the tricyclic antidepressant includes imipramine pamoate (5-[3-(dimethylamino) propyl]-10,11-dihydro-5H-dibenzazepine 4,4-methlyenebis-(3-hydroxy-2-napthoate) (2:1 ratio of pamoate to imipramine). Imipramine pamoate is commercially available as TOFRANIL-PM from Mallinckrodt Inc. in capsules of 75 mg, 100 mg, 125 mg and 150 mg.

For an adult, a total daily dosage of imipramine in a medicine according to an embodiment of the present invention may be in a range of from about 5 mg/day to about 100 mg/day, about 10 mg/day to about 25 mg/day, about 25 mg/day to about 50 mg/day, or about 50 mg/day to about 75 mg/day. Here and elsewhere, range limitations may be combined, for example, a range may be from about 10 mg/day to about 50 mg/day.

In one embodiment, the total daily dosage of the tricyclic antidepressant for a given patient is lower than the effective psychiatric total daily dosage for that patient, if the patient were being treated for a psychiatric condition. In one embodiment, each individual dose of the tricyclic antidepressant for a given patient is lower than the effective psychiatric individual dose for that patient, if the patient were being treated for a psychiatric condition. That is, in such embodiments, the dosages given to any individual patient are lower than would be used if the patient were being treated for a psychiatric condition. By “psychiatric condition” is meant any treatment such as an anti-depressant treatment, whether administered by a psychiatric specialist, general practitioner or other medical or psychological professional.

Alternatively, in one embodiment the total daily dosage may be based on patient weight. According to an embodiment of the present invention a total daily dosage of imipramine in a medicine may be in a range of from about 0.1 milligram/kilogram body weight/day (mg/kg/day) to about 2.5 mg/kg/day, about 0.2 mg/kg/day to about 1.2 mg/kg/day, about 0.5 mg/kg/day to about 2.0 mg/kg/day, about 0.5 mg/kg/day to about 0.75 mg/kg/day, about 0.75 mg/kg/day to about 1.25 mg/kg/day, or about 1.25 mg/kg/day to about 2.0 mg/kg/day.

For elderly, infirm, or smaller than average-sized patients a total daily dosage amount may be adjusted downward. In one embodiment, for example, the total daily dosage may be in a range of from about 5 mg to about 30 mg, or about 5 mg to about 10 mg.

Because children may have a relatively higher glomerular filtration rate (GFR) the dosage may need to be adjusted upward to accommodate such, rather than adjusted downward as seen in anti-depression treatment. In one embodiment, a child dosage may be up to four times greater than the adult dosages, or up to about 300 mg/day.

The dosage amounts may be affected by several factors. Such factors may include the type and nature of concurrently taken medications. For example, relatively decreased dosages of, for example, imipramine pamoate may have a comparable effect to higher dosages in the presence of metabolism inhibiting compositions. Metabolism inhibiting compositions may include methylphenidate HCI (which is commercially available from Ciba-Geigy Corporation (Basel, Switzerland), a division of Novartis Pharmaceuticals Corporation, under the trade names of RITALIN and RITALIN SR).

The total daily dosage need not be taken at one time. Rather, the dosage may be taken as portions of the total daily dosage over the course of the day. For example, a 75 mg/day dose may be taken as 25 mg three times a day, optionally each with a meal.

Stool softener, as used herein, is distinguished from laxatives. Laxatives may include bulk, osmotic and stimulant-type. Bulk laxatives include soluble and insoluble fiber. Soluble fiber can include psyllium husks and is commercially available as METAMUCIL from Procter & Gamble Inc. (Cincinnati, Ohio). Insoluble fiber can include wheat bran. Osmotic laxatives are not absorbed and function by pulling water into the colon via osmotic action (e.g., magnesium hydroxide, such as PHILLIP'S MILK OF MAGNESIA, which is commercially available from Bayer Corporation (Pittsburgh, Pa.)). Stimulant laxatives interfere with absorption of water from the colon lumen and motility of fecal material therethrough.

By way of contrast, a stool softener acts to emulsify water and/or oil into fecal matter and thus soften the consistency. A fecal lubricant acts by lubricating the fecal matter and allowing it to pass though the colon with a reduced amount of friction. Suitable stool softeners include surfactants, such as anionic surfactants, nonionic surfactants, cationic surfactants, and amphoteric surfactants which include both an anionic and a cationic portion connected by a covalent bond. Suitable surfactants include anionic surfactants. In one embodiment, the stool softener includes bis (2-ethylhexyl) sulfosuccinate sodium salt (“docusate sodium”), which is commercially available from Purdue Pharma L.P. (Stamford, Conn.) as COLACE. The manufacturer recommended initial dosage level for COLACE is usually about 50-200 mg/day in divided doses. Docusate sodium has the following structure:

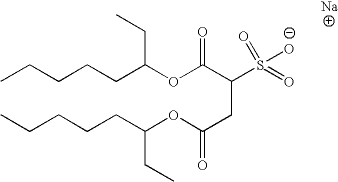

Other suitable metal salts of sulfosuccinate may be useful, and the metal may be potassium, calcium and the like. PERICOLACE (which is a tradename for docusate plus casanthrol), sodium dodecylsulfate (SDS), sodium cholate, sodium deoxycholate (DOC), N-lauroylsarcosine sodium salt, lauryldimethylamine-oxide (LDAO), and cetyltrimethyl ammonium bromide (CTAB) may be used in embodiments according to the invention.

The fecal lubricant may include, for example, commercially available mineral oil or liquid paraffin. The stool softener and fecal lubricant may be used alone and in combination with each other. In combination, the stool softener can emulsify the fecal lubricant into the stool.

In one embodiment according to the invention, the stool softener may be used in efficacious amounts at dosage levels of less than 200 mg/day. In one embodiment, the dosage of stool softener may be greater than 200 mg/day, and may be used in an amount of up to about 300 mg/day, or up to about 400 mg/day. Alternatively, the amount of the stool softener may be determined with reference to body weight. In one embodiment, the total daily dosage may be in a range of from about 1 mg/kg/day to about 4 mg/kg/day. In one embodiment, the total daily dosage may be in a range of from about 1.0 mg/kg/day to about 2.0 mg/kg/day, from about 2.0 mg/kg/day to about 3.0 mg/kg/day, or from about 3.0 mg/kg/day to about 4.0 mg/kg/day.

The frequency, and amount, of stool softener dosages may be determined on an individual basis. However in one embodiment, the daily dosage may be 300 mg/day taken in three 100 mg doses spaced over the course of the day, optionally with a meal. As noted above the stool softener may be taken concomitant with the tricyclic antidepressant or may be taken at a different time relative to the tricyclic antidepressant. The regimen for taking the medicine, or components or portions thereof, is discussed further below.

The dosage amount of tricyclic antidepressant to stool softener may be expressed as a ratio or a proportion. In one embodiment, the ratio of tricyclic antidepressant to stool softener is in a range of from about 1:80 to about 3:1, from about 1:12 to about 1:6, from about 1:4 to about 1:3, from about 1:2 to about 1:1, or from about 2:1 to about 3:1. In one embodiment, the ratio may be preselected based on weight, symptom severity, symptom type, symptom frequency, dietary considerations, type of tricyclic antidepressant and stool softener, dose regimen, administration method, environmental considerations, other or additional medications, and the like. In one embodiment, the ratio may be selected based on individual responsiveness, dietary considerations, environmental considerations, side effects, aggravating conditions such as stress level, other or additional medications, and the like.

The components of the medicine need not be taken at the same time as each other. For example, in one embodiment the tricyclic antidepressant may be taken concomitant or concurrent with the stool softener, and in one embodiment the tricyclic antidepressant may be taken at a time different than the stool softener.

The administration method may be selected with reference to the form, packaging and configuration of the tricyclic antidepressant and the stool softener. The regimen for taking the medicine, or components or portions thereof, is discussed further below.

With reference to form of the medicine, at least a portion of the medicine may be a pill, capsule, gelcap, a coated or chewable tablet, a chewable gum, an ingestible liquid admixture, transdermal patch, an inhalable powder or mist, an enema or suppository, an intravenous solution or an intramuscular injectable liquid.

Administration of the dose may include selecting an entry method or application based on the form of the medicine. For example, imipramine may be formed as a gelcap, and sodium docusate may be an ingestible liquid, the imipramine may be swallowed and the sodium docusate may be imbibed or drank. In one embodiment, imipramine and sodium docusate may be combined or commingled in a single capsule.

In one embodiment, imipramine and sodium docusate may be combined or commingled as portions contained in a plurality of capsules. The portions may be fractional amounts of the total daily dose. The plurality of capsules may be taken throughout the course of the day to distribute the medicine over the course of the day. For example, the medicine may be in the form of pills that each containing 50 mg of stool softener admixed with 5 mg of tricyclic antidepressant. The total daily dose may be 150 mg of stool softener and 15 mg of tricyclic antidepressant. Taking three doses of the portions over the course of a day would enable the total daily dosage to be achieved.

Similarly, the total daily dosage amount may be controlled by selecting portions containing fractional dosage amounts. For example, the total daily dosage amount may be adjusted from 150 mg of stool softener and 15 mg of tricyclic antidepressant per day to 300 mg of stool softener and 30 mg of tricyclic antidepressant per day. Taking six pills each containing 50 mg of stool softener admixed with 5 mg of tricyclic antidepressant would achieve the adjusted total daily dosage.

The number of fractional doses or portions taken per day may be adjusted to correspond to preselected factors. Such factors may include, for example, seasonal changes (e.g., dehydration, being more prevalent in summer months, may result in a temporary amelioration of fecal incontinence), aging, the natural course of the gastrointestinal disorder, stress inducing situations, and others that may affect the occurrence or severity of symptoms of the gastrointestinal disorder.

Suitable forms of the medicine, such as pills, capsules, gelcaps, tablets, and the like, may be packaged in multi-dose packages, such as blister packs. Thus, the blister pack may contain dosages of a medicine according to the present invention.

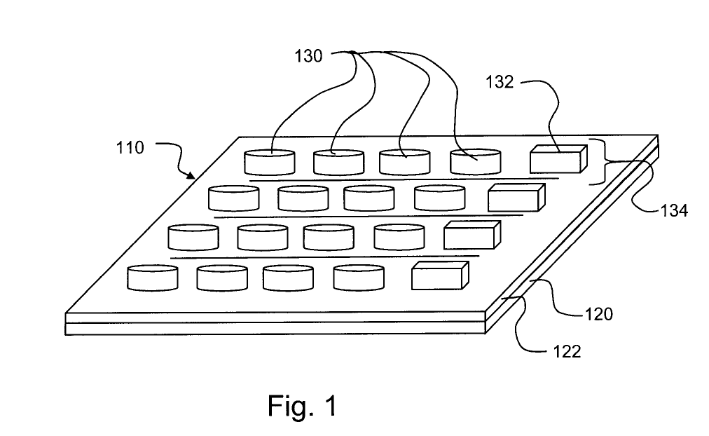

With reference to FIG. 1, a packaged treatment regimen 100 showing an embodiment according to the invention includes a blister pack 110. The blister pack 110 has a base layer 120 secured to a bottom surface of a top layer 122. The top layer 122 defines storage blisters, and the base layer 120 can operate to seal the blisters to releasably contain doses of the medicine, or portions of the medicine. The blisters in the illustrated embodiment define differing shapes merely for the purpose of ease of differentiation.

In the embodiment shown, the stool softener is housed in the blisters labeled 130, and the tricyclic antidepressant is housed in the blister labeled 132. A row or strip 134 may include an amount equivalent to a preselected total daily dose of the medicine. The illustrated embodiment shows a total daily dose that includes four portions of stool softener (at, for example, 75 mg each) and one portion of tricyclic antidepressant (at, for example, 25 mg) that are physically separated from each other. Correspondingly, there are four blisters 130 for housing the stool softener and one blister 132 for housing the tricyclic antidepressant. Portions are thus provided so that the stool softener may be taken four times a day for 300 mg/day total daily dose, and the tricyclic antidepressant may be taken once a day for 25 mg/day total daily dose. Furthermore, the tricyclic antidepressant may be taken with one of the stool softener doses or at another time, as desired.

The strip 134 is one of four shown on the blister package 110, which is a four day supply of medicine. The blister package 110 may have instructions printed thereon that indicate what the dosage regimen may be, and, optionally and/or additionally, directions for varying portion dosage with reference to symptomology or exacerbating conditions.

Naturally, in other embodiments (not shown) the tricyclic antidepressant and stool softener portions may include dosages having differing amounts for different total daily dosages, may have differing numbers of doses for the same or different total daily dosages, and may have doses that include both the tricyclic antidepressant and the stool softener in a single form (such as a pill containing both the tricyclic antidepressant and the stool softener). Further, other embodiments according to the invention may have the tricyclic antidepressant and/or the stool softener in a form other than pill, gel cap, and the like, and may not be amenable to blister packaging. Suitable packaging may then be selected based on the form of the tricyclic antidepressant and the stool softener, and whether the tricyclic antidepressant and the stool softener are admixed or physically separate.

With reference to forms of the medicine other than those discussed above, the ingestible liquid admixture may be administered in pre-measured amounts. The transdermal patch, the chewable gum, the intravenous solution, or the intramuscular injectable liquid, and the oral and/or nasal inhaler (for the inhalable powder or mist) may be used to deliver the tricyclic antidepressant, while the stool softener may be administered via a different method. The enema or suppository may contain the stool softener and may be administered in a conventional manner. For orally administrable embodiments in which at least one component or portion of the medicine is taken orally, masking agents may be used. For example, edible carriers, such as food, may be used to enhance palatability of the medicine or medicine component. In one embodiment, the food is selected to have a pharmacological effect. For example, prune juice has a known tendency to increase bowel movement frequency, and this tendency may be factored into the dosage amounts for the medicine or medicine components.

In one embodiment, the medicine may contain additional material either admixed or separate from the tricyclic antidepressant, the stool softener, or both. For example, the medicine may contain a skeletal muscle relaxant, a narcotic, or a proton pump inhibitor, and may further include a suitable pharmaceutical excipient, diluent, or carrier selected with regard to the intended route of administration and standard pharmaceutical practice. Suitable skeletal muscle relaxants include cyclobenzaprine hydrochloride, which may be classified as a tricyclic antidepressant, is commercially available from McNeil Corporation (Fort Washington, Pa.) as FLEXERIL. cyclobenzaprine hydrochloride may be combined in the medicine according to the invention. A useful dose of cyclobenzaprine hydrochloride may be 10 milligrams 4 times a day. A dosage upper limit may be about 40 milligrams a day.

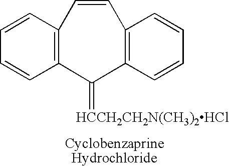

Suitable narcotics include opioid agonists such as PERCOCET (oxycondone plus acetaminophen), which is commercially available from Endo Laboratories, Inc. (Chadds Ford, Pa.). Suitable proton pump inhibitors include omeprazole or 5-methoxy-2-[[(4-methoxy-3,5-dimethyl-2-pyridinyl) methyl] sulfinyl]-1H-benzimidazole, which is commercially available from AstraZeneca LP (Wilmington, Del.) as PRILOSEC, and lansoprazole, which is commercially available from TAP Pharmaceutical Products Inc. (Lake Forrest, Ill.) as PREVACID.

In one embodiment, the medicine further includes a beta-blocker, such as atenolol, which is commercially available from Medley Pharmaceuticals, Ltd (Maharashtra, India) as TENORMIN. Atenolol is a synthetic, betal-selective (cardioselective) adrenoreceptor blocking agent or “beta-blocker”, that may be chemically described as benzeneacetamide, 4-[2′-hydroxy-3′-[(1-methylethyl) amino] propoxy] benzeneacetamide. Atenolol may block the action of the sympathetic nervous system. Because the sympathetic nervous system controls or influences the pace of the heart beat, blocking the action of these nerves can reduce the heart rate. Atenolol may reduce the force of heart muscle contraction, lower blood pressure, and affect bowel movement frequency. Where tachycardia may be caused, for example, as a result of the action of the tricyclic antidepressant, a beta-blocker such as atenolol may be used to maintain the heart rate in a desired range.

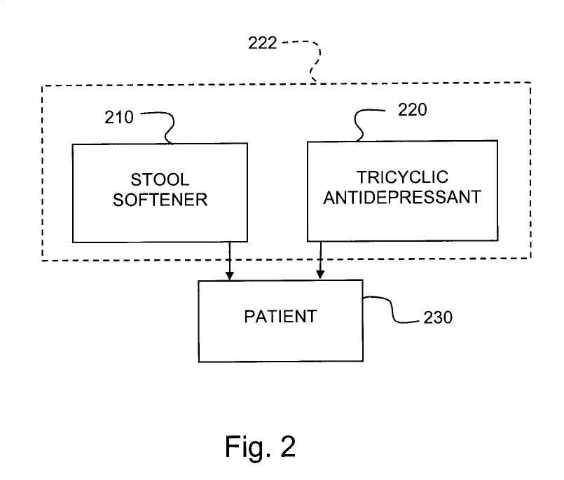

With reference to FIG. 2, a method according to the present invention as a block diagram 200. A stool softener 210 and a tricyclic antidepressant 220 comprise a medicine 222. The stool softener 210 and the tricyclic antidepressant 220 are administered to a patient 230 suffering from a gastrointestinal disorder.

In patients who have had a colostomy or the stool bypasses the colon, the consistency of the stool may sometimes be undesirable. Sometimes a colostomy bag collects the stool using a stoma. Stool having an undesirable consistency may be difficult to handle and may irritate the skin adjacent the stoma because the fecal material has not been processed yet through the colon. Administration of the medicine according to the invention may improve the consistency of the fecal material. The small bowel may process the fecal material to a consistency similarly to a consistency of fecal material that had been processed by the colon. In one embodiment, in response to the administration of the medicine, the water content is reduced for fecal material entering a colostomy bag from a patient who has had a colostomy, and the fecal material may be emulsified with the water. The reduced water content, alone or in conjunction with the emulsification, may improve the consistency of the fecal material and the ease of handling of the fecal material.

### EXAMPLES

Embodiments according to the invention are illustrated in the following examples. More particularly, the treatment of fecal incontinence by methods and with medicines according to the present invention is shown.

### Example 1

An ambulatory Caucasian female patient, 28 years old, presents with sprain lumbar spine. The spinal injury relates to back pain, with spasms, stress urinary incontinence and stress bowel or fecal incontinence. For example, a sneeze may result in dual bladder and bowel incontinence. On presentment, protective pads are worn to absorb urine and feces.

The patient is treated with a daily dose of medicine, which includes 75 mg of imipramine pamoate and a stool softener. After several days of daily treatment via oral administration, the patient notes improvement of stress bowel incontinence. The patient stops using protective absorbent pads. The patient tolerates a dry mouth.

### Example 2

An ambulatory Caucasian male patient, 53 years old, presents with a history of tib-fib fracture of his right leg, as well as a back or spinal injury. The patient may have experienced several cerebrovascular accidents (CVA) of unknown etiology within the two years previous. The patient experiences a gradual onset of fecal incontinence with no known precipitating event.

The patient is treated with a daily dose of medicine, which includes 75 mg of imipramine pamoate and a stool softener. The patient takes TYLOX, oxycontin, neurotin, aspirin, and anti-coagulants. After several days of daily treatment via oral administration, the patient notes control of fecal incontinence, but bowel urgency remains. The patient is able to stop using protective absorbent pads. The patient tolerates a dry mouth and dry eyes.

### Example 3

An ambulatory Caucasian female patient, 43 years old, presents with sprain lumbar spine. The spinal injury relates to back pain, with spasms, urgency and incontinence of the bladder and bowel.

The patient is treated with a daily dose of medicine, which includes 75 mg of imipramine pamoate. After several days of daily treatment via oral administration, the patient notes full control of bladder and bowel functions. The patient develops constipation and is prescribed docusate sodium (COLACE) in conjunction with the imipramine pamoate. The constipation is relieved by the docusate sodium.

The patient stops taking the daily dosages. After about three days without treatment, the bowel and bladder urgency and incontinence recur.

### Example 4

An ambulatory Caucasian male patient, 45 years old, presents with sprain of sacrum, lumbar disc displacement, sprain lumbosacral, recurrent depression (psych-severe), and gastritis. The complaints include pain, spasms, depression, upset stomach, irritable bowel syndrome, and fecal and urinary incontinence (awake and sleeping).

The patient is treated with a total daily dose of medicine, which includes 75 mg of imipramine hydrochloride (25 mg/3 times daily) and a stool softener. After several days of daily treatment via oral administration, the patient notes full control of bladder and bowel functions (awake and sleeping). The patient tolerates a dry mouth. The patient switches to 75 mg/day (1 dosage/day) of imipramine pamoate, and a stool softener, with continued full control of bowel functions (awake and sleeping).

### Example 5

An ambulatory male patient presents with a nerve injury to the spine. The complaints include fecal incontinence (awake and sleeping).

The patient is treated with a total daily dose of medicine, which includes 25 mg of tricyclic antidepressant (imipramine hydrochloride) and 300 mg of stool softener (docusate sodium). After several days of daily treatment via oral administration, the patient notes partial to full control of bowel functions (awake and sleeping). That is, a reduction or elimination of fecal incontinence.

### Example 6

An ambulatory female patient presents with a pelvic nerve injury. The complaints include chronic, intermittent fecal incontinence.

The patient is treated with a total daily dose of medicine, which includes 25 mg of imipramine hydrochloride and 300 mg of stool softener (docusate sodium), ingested separately. After several days of daily treatment via oral administration, the patient notes partial to full control of bowel functions (awake and sleeping). That is, a reduction or elimination of fecal incontinence.

### Example 7

An ambulatory male patient presents with a compromised vascular supply to the bowel. The complaints include chronic, intermittent fecal incontinence.

The patient is treated with a total daily dose of medicine, which includes an admixture of 5 mg of imipramine hydrochloride and 250 mg of stool softener (docusate sodium). After several days of daily treatment via oral administration, the patient notes partial to full control of bowel functions (awake and sleeping). That is, a reduction or elimination of fecal incontinence. A selective beta-blocker is administered in response to tachycardia on an as-needed basis.

The processes and embodiments described herein are examples of compositions, systems and methods having elements corresponding to the elements of the invention recited in the claims. This written description may enable those skilled in the art to make and use embodiments having alternative elements that likewise correspond to the elements of the invention recited in the claims. The intended scope of the invention thus includes other compositions, systems and methods that do not differ from the literal language of the claims, and further includes other compositions, systems and methods that are equivalent to, or have insubstantial differences from, the literal language of the claims.

What is claimed is:

1. A method of treating an ambulatory patient having a condition comprising fecal incontinence, fecal urgency, or a combination thereof, the method comprising:
administering a dose of a medicine to the patient, the medicine comprising
a tricyclic antidepressant, and
a stool softener,
wherein the condition is a result of one or more of a nerve injury, a course of radiation treatment, a hemorrhoid surgery, a chemotherapy treatment, a compromised vascular supply to the bowel, malnutrition, diabetes, or cancer.

2. The method as defined in claim 1, wherein the nerve injury is a spinal nerve injury, a spinal cord injury, or a pelvic nerve injury.

3. The method as defined in claim 1, wherein the tricyclic antidepressant is present in the medicine in an amount efficacious for the condition but which would not be efficacious for a psychiatric condition in the patient.

4. The method as defined in claim 1, wherein the tricyclic antidepressant is present in an efficacious amount in a range from about 5 mg/day to about 100 mg/day.

5. The method as defined in claim 1, wherein the stool softener is present in an amount in a range of greater than about 200 milligrams per day.

6. The method as defined in claim 1, wherein the tricyclic antidepressant comprises imipramine hydrochloride, imipramine pamoate, a pharmacologically acceptable salt of imipramine, or combinations of two or more thereof.

7. The method as defined in claim 1, wherein the patient is a non-elderly adult patient.

8. The method as defined in claim 1, wherein the patient is not clinically depressed.

9. The method as defined in claim 1, wherein the stool softener comprises a surfactant, a fecal lubricant, or a combination thereof.

10. The method as defined in claim 9, wherein the surfactant comprises an anionic surfactant.

11. The method as defined in claim 10, wherein the anionic surfactant comprises docusate sodium.

12. The method as defined in claim 1, wherein the condition is a chronic condition, and further comprising administering the medicine over an extended period of time corresponding to a treatment of the chronic condition.

13. The method as defined in claim 1, further comprising varying amounts of the tricyclic antidepressant and the stool softener over a course of treatment in response to preselected factors.

14. The method as defined in claim 1, further comprising administering to the patient a beta-blocker that is effective to reduce tachycardia, a skeletal muscle relaxant, a narcotic, a proton pump inhibitor, or two or more thereof.

15. The method as defined in claim 1, wherein the tricyclic antidepressant to stool softener ratio is in a range of from about 1:80 to about 3:1.

16. A method for treating a gastrointestinal disorder in an ambulatory patient, comprising:
causing an interaction with muscarinic receptors in the patient to reduce or eliminate fecal incontinence of a stool, fecal urgency, or both of the patient, and one or both of:
delivering into fecal matter of the patient an oil and water by emulsification using a surfactant to soften the stool of the patient, and
delivering into fecal matter of the patient a fecal lubricant to facilitate passage of the stool,
wherein the delivering occurs in the bowel of the patient substantially simultaneously or temporally adjacent to the interaction with the muscarinic receptors,
wherein the gastrointestinal disorder is a result of one or more of a nerve injury, a course of radiation treatment, a hemorrhoid surgery, a chemotherapy treatment, a compromised vascular supply to the bowel, malnutrition, diabetes, or cancer.

17. A method of treating an ambulatory non-elderly adult patient having a gastrointestinal disorder comprising fecal incontinence, fecal urgency, or a combination thereof, the method comprising administering a dose of a medicine to the patient having the gastrointestinal disorder, the medicine comprising
a tricyclic antidepressant in an amount efficacious for the condition but which would not be efficacious for a psychiatric condition in the patient, and
a stool softener comprising a surfactant, a fecal lubricant, or a combination thereof.

18. The method as defined in claim 17, wherein the stool softener is present in an amount in a range of greater than about 200 milligrams per day.

19. The method as defined in claim 17, wherein the tricyclic antidepressant comprises imipramine hydrochloride, imipramine pamoate, a pharmacologically acceptable salt of imipramine, or combinations of two or more thereof.

20. The method as defined in claim 17, wherein the surfactant comprises an anionic surfactant.
